(19) United States
(12) Patent Application Publication
(10) Pub. No.: US 2007/0162111 A1
(43) Pub. Date: Jul. 12, 2007

(54) Apparatus and method for replacing a cardiac valve

(75) Inventors: Kiyotaka Fukamachi, Mayfield Heights, OH (US); Raymond Dessoffy, Parma, OH (US)

Correspondence Address: TAROLLI, SUNDHEIM, COVELL & TUMMINO L.L.P., 1300 EAST NINTH STREET, SUITE 1700, CLEVEVLAND, OH, 44114, US

(73) Assignee: The Cleveland Clinic Foundation

(21) Appl. No.: 11/481,459

(22) Filed: Jul. 6, 2006

Related U.S. Application Data
(60) Provisional application No. 60/696,934, filed on Jul. 6, 2005.

(51) Int. Cl. A61F 2/24

(52) U.S. Cl. 623/2.11; 623/2.38

(57) ABSTRACT

An apparatus for replacing a cardiac valve includes a first ring assembly and a second ring assembly. The first ring assembly includes a first magnetic member. The second ring assembly includes a second magnetic member and is magnetically attachable to the first magnetic member to sealingly attach the first and second ring assemblies together. A prosthetic cardiac valve is secured within the second ring assembly. A method for replacing a cardiac valve is also described.

### RELATED PATENT APPLICATION

This application claims priority to the filing date of U.S. Provisional Application No. 60/696,934, filed Jul. 6, 2005.

### TECHNICAL FIELD

The present invention relates generally to prosthetic cardiac devices, and more particularly to an apparatus and method for replacing a cardiac valve.

### BACKGROUND OF THE INVENTION

Implantable heart valve prostheses have been used to replace various diseased or damaged natural aortic valves, mitral valves, pulmonary valves and tricuspid valves of the heart. The aortic and mitral valves are most frequently replaced due to heart disease, congenital defects and/or injury. Diseased or malfunctioning heart valves are typically replaced with either mechanical or bioprosthetic heart valve prostheses. A typical known bioprosthetic valve 100 is depicted in place within a valve annulus 102 in FIGS. 1 and 2. Bioprosthetic valves, such as that shown in FIGS. 1 and 2, are typically made of biological material, such as bovine pericardial tissue, and are particularly vulnerable to structural degeneration. Consequently, reoperation in patients with a bioprosthetic valve is often done to replace the original bioprosthetic valve with a new bioprosthetic valve.

Reoperations for bioprosthetic cardiac valve failure are associated with significant mortality and morbidity, and can pose formidable technical challenges. For instance, reoperations on the mitral valve can be associated with cardiac rupture at the atrioventricular junction or posterior ventricular wall where a strut may be embedded. Known replaceable bioprosthetic valves have attachment interfaces which are subject to thrombosis formation. Mitral valve reoperations can also result in damage to the left circumflex coronary artery during removal of the degenerated bioprosthesis and insertion of a new bioprosthesis. Further, with removal and replacement of either the mitral or aortic valve, late perivalvular leaks may also develop.

### SUMMARY OF THE INVENTION

In an embodiment of the present invention, an apparatus for replacing a cardiac valve is described. A first ring assembly includes a first magnetic member. A second ring assembly includes a second magnetic member and is magnetically attachable to the first magnetic member to sealingly attach the first and second ring assemblies together. A prosthetic cardiac valve is secured within the second ring assembly.

In an embodiment of the present invention, an apparatus for replacing a cardiac valve having a valve annulus is described. The valve annulus has a superior aspect and an inferior aspect. A first ring assembly includes a first magnetic member and is attachable to the valve annulus. A second ring assembly includes a second magnetic member and is magnetically attachable to the first magnetic member to sealingly attach the first and second ring assemblies together. A prosthetic cardiac valve is secured within the second ring assembly.

In an embodiment of the present invention, a method for replacing a native cardiac valve within a valve annulus is described. The native cardiac valve is removed from the valve annulus. A first ring assembly is attachable to the valve annulus. The first ring assembly includes a first magnetic member. A second ring assembly includes a second magnetic member. The second ring assembly includes a prosthetic cardiac valve secured thereto. The first ring assembly is secured to the valve annulus. The second ring assembly is moved into magnetic engagement with the first ring assembly to position the prosthetic cardiac valve into a predetermined relationship within the valve annulus.

### BRIEF DESCRIPTION OF THE DRAWINGS

For a better understanding of the invention, reference may be made to the accompanying drawings, in which:

FIG. 1 is a perspective view of a known bioprosthetic valve;

FIG. 2 is a perspective sectional view similar to FIG. 1;

FIG. 3 is a partial sectional view of a human heart;

FIG. 4 is an exploded perspective view of an embodiment of the present invention;

FIG. 5A is a sectional view taken along line 5A-5A of FIG. 4;

FIG. 5B is a sectional view taken along line 5B-5B of FIG. 4;

FIG. 6 is a partial sectional, perspective view of the embodiment within a human heart;

FIG. 7A is a partial perspective view of the embodiment;

FIG. 7B is a partial perspective view of the embodiment;

FIG. 8A is a sectional schematic view of the embodiment of FIG. 4 in an assembled state within a human heart;

FIG. 8B is a sectional schematic view of the embodiment of FIG. 4 in an alternate configuration within a human heart;

FIG. 9 is a partial sectional view of the embodiment of FIG. 8A in an assembled state within a human heart;

FIG. 10A is a partial schematic view of a separation tool for use with any embodiment of the present invention;

FIG. 10B is a perspective view of the separation tool of FIG. 10A in a first mode of operation;

FIG. 10C is a perspective view of the separation tool of FIG. 10A in a second mode of operation;

FIG. 11A is a perspective view of a procedure for using any embodiment of the present invention; and

FIG. 11B is a perspective view of a procedure for using any embodiment of the present invention.

### DETAILED DESCRIPTION

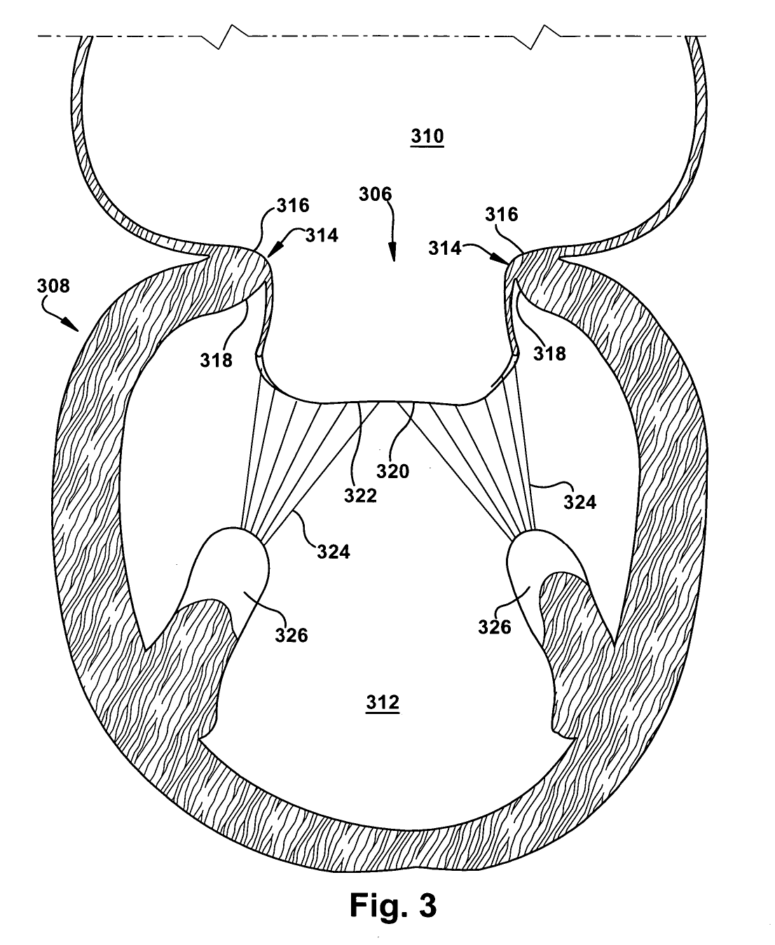

FIG. 3 depicts the positioning of the native mitral valve 306 within the heart 308. The mitral valve 306 is located between the left atrium 310 and the left ventricle 312, and functions to prevent backflow of blood from the left ventricle into the left atrium during left ventricular contraction. The mitral valve 306 has a D-shaped annulus 314 with superior and inferior aspects 316 and 318, respectively, which define the opening between the left atrium 310 and the left ventricle 312. The mitral valve 306 is formed by two leaflets; namely, the anterior leaflet 320 and the posterior leaflet 322. The anterior leaflet 320 extends along the generally planar base of the D-shaped annulus 314, while the posterior leaflet 322 extends arcuately around the curved portion of the D-shaped annulus of the mitral valve 306. Chordae tendineae 324 extend between the free edges of both leaflets 320 and 322 and the papillary muscles 326 in the left ventricle 312. The leaflets 320 and 322 are excised from the native valve annulus 314 or the native mitral valve 306 is otherwise inactivated or removed before being replaced with the present invention.

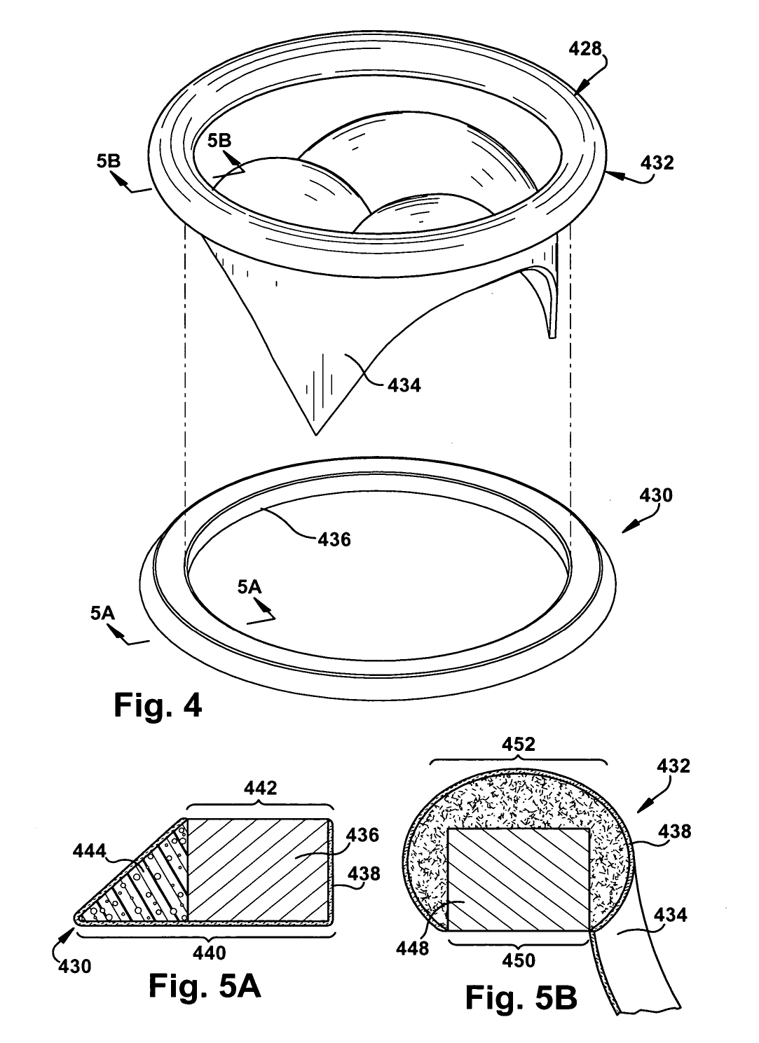

In accordance with an embodiment of the present invention, FIG. 4 depicts an exploded view of an apparatus 428 for replacing a native cardiac valve. The apparatus 428 will be discussed herein as replacing a native mitral valve. It should be understood, however, that the apparatus 428 could also or instead be used to replace other cardiac valves, such as the tricuspid, aortic, and pulmonary valves.

As shown in FIG. 4, the apparatus 428 for replacing the native mitral valve 306 comprises a first ring assembly 430, and a second ring assembly 432 including a prosthetic cardiac valve 434. The first ring assembly 430 includes a first magnetic member 436 at least partially covered by a biocompatible material 438 (see FIG. 5A). The first ring assembly 430 can be secured into position in the superior aspect 316 of the annulus 314 of the native mitral valve 306.

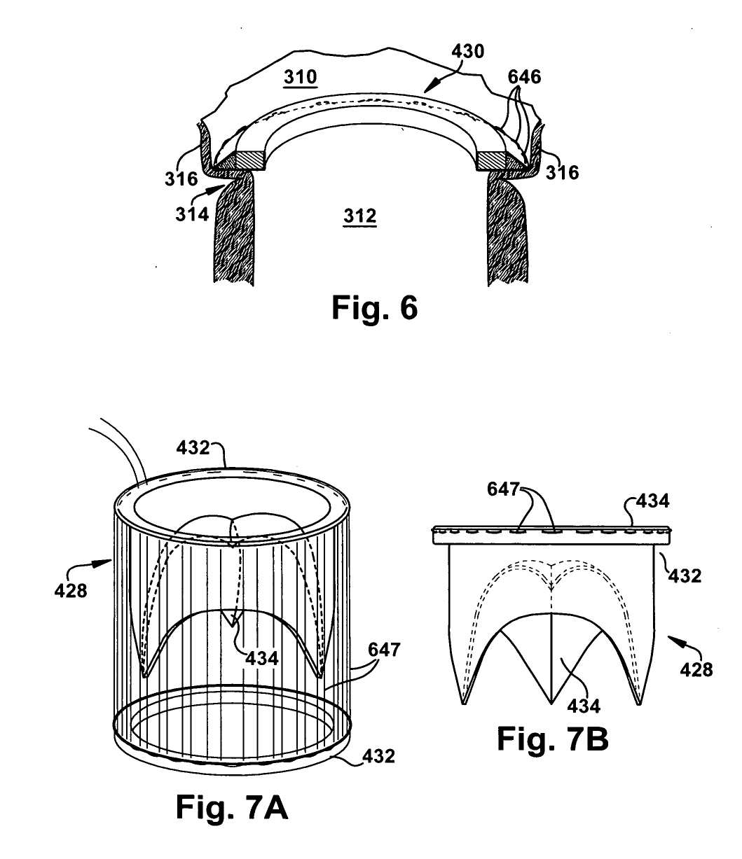

The first ring assembly 430 may have a generally annular shape and include first and second aspects 440 and 442. As shown in the cross-section of FIG. 5A, the first aspect 440 of the first ring assembly 430 is adapted to conform to the superior aspect 316 of the annulus 314 of the native mitral valve 306 (this mating shown in partial cross-section in FIG. 6). For instance, the first aspect 440 of the first ring assembly 340 may have a concave cross-sectional shape (not shown) for conforming to the convex shape of the annulus 314 of the native mitral valve 306. The second aspect 442 of the first ring assembly 430 has a shape complementary to the shape of the second ring assembly 432 to adapt the second aspect for mating to the second ring assembly.

As shown in the cross-sectional view of FIG. 5A, the first ring assembly 430 may also include at least one sewing ring 444 operatively attached to the first magnetic member 436. The sewing ring 444, when present, may supplement the shape of the first ring assembly 430 to reinforce the first magnetic member 436 or otherwise adapt the first ring assembly for mating with the second ring assembly 432. For instance, if the first magnetic member 436 includes a plurality of small magnets (not shown) arrayed around the first ring assembly 430, the sewing ring 444 could maintain the small magnets in the desired arrangement for mating with the second ring assembly 432. The sewing ring 444 may also or instead supplement the shape of the first ring assembly 430 to adapt the first ring assembly for mating with the superior aspect 316 of the annulus 314 of the native mitral valve 306. For example, the sewing ring 444 may operate to conform the first ring assembly 430 to an undulating contour (not shown) of the annulus 314.

The sewing ring 444 may also or instead be used to anchor the first ring assembly 430 to the annulus 314 of the native mitral valve 306. For instance, the first ring assembly 430 may be attached to the annulus 314 by a plurality of sutures 646 (shown in FIG. 6) threaded through the sewing ring 444 to stitch the first ring assembly to the annulus 314 of the native mitral valve 306. The sewing ring 444 may be comprised of any suitable material, which can readily be chosen by one of ordinary skill in the art for a particular application of the present invention. For example, a flexible, resiliently yieldable material such as silicon, plastic, polytetrafluoroethylene (PTFE), expanded-PTFE (ePTFE), polyurethane, or other similar material could be chosen if the sewing ring 444 is to be stitched to the annulus 314 with sutures 646.

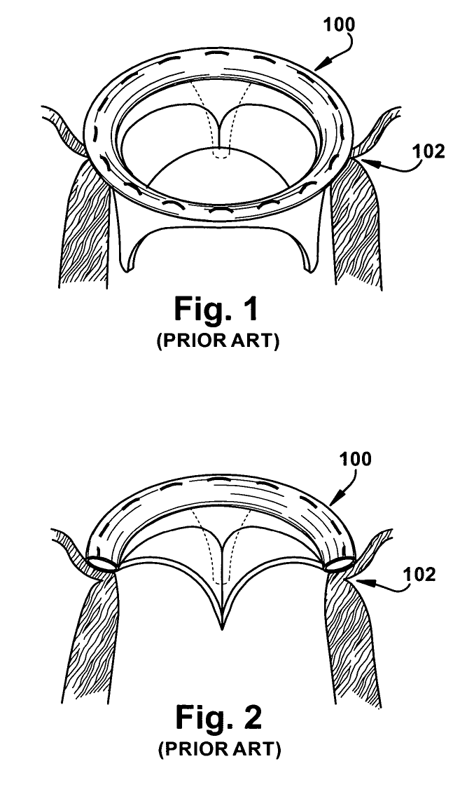

As shown in FIG. 4, a suitable prosthetic cardiac valve 434 is secured within the second ring assembly 432 in any desired manner. For instance, and as shown in the sequence of FIGS. 7A and 7B, the prosthetic cardiac valve 434 may be fastened to other structures of the second ring assembly 432 with sutures 647. Prosthetic cardiac valves 434 are well known in the art, and may have similar features to those of the known bioprosthetic valve 100 shown in FIGS. 1 and 2. The prosthetic cardiac valve 434 of the present invention may be a mechanical valve, a bioprosthetic valve, a combination thereof, or any other suitable valve. Mechanical heart valves are made from materials of synthetic origin, like metals (e.g., stainless steel and molybdenum alloys), ceramics, and polymers. Mechanical heart valves typically utilize a ball, a disc, valve leaflets, or other mechanical valving devices to regulate the direction of blood flow through the prosthesis. Specific examples of mechanical heart valves are known in the art.

In addition to, or instead of, synthetic materials, materials of biological origin (e.g., animal pericardial tissue or other animal, human, or laboratory-grown tissues) are typically used to construct bioprosthetic heart valves. For instance, the bioprosthetic cardiac valve 434 of the present invention may be made from one or more pieces of biological material formed into a bi-leaflet conduit having dimensions that correspond to the dimensions of the native mitral valve 306. Specific examples of bioprosthetic heart valves are known in the art.

The second ring assembly 432 of the apparatus 428 comprises a second magnetic member 448 at least partially covered with a biocompatible material 438, as shown in FIG. 5B. The second magnetic member 448 is magnetically attracted to the first magnetic member 436 of the first ring assembly 430. This magnetic attraction sealingly attaches the second ring assembly 432 to the first ring assembly 430 adjacent the superior aspect 316 of the annulus 314 of the native mitral valve 306. A “sealing” attachment does not require a completely impermeable interface; a substantially fluidtight connection will suffice. The first and second magnetic members 436 and 448 are shown in the Figures as having rectangular cross-sections. However, like any structure of the present invention, the first and second magnetic members 436 and 448 may have any suitable cross-section and need not be identically shaped.

As shown in the cross-sectional view of FIG. 5B, the second ring assembly 432 includes first and second aspects 450 and 452, respectively. The second ring assembly 432 may have a generally annular shape and may be adapted to mate with the first ring assembly 430. In particular, the first and second aspects 450 and 452 of the second ring assembly 432 are respectively adapted to mate with the first ring assembly 430 and to accept the prosthetic cardiac valve 434. For instance, the first aspect 450 of the second ring assembly 432 may have a shape complementary to the shape of the second aspect 442 of the first ring assembly 430.

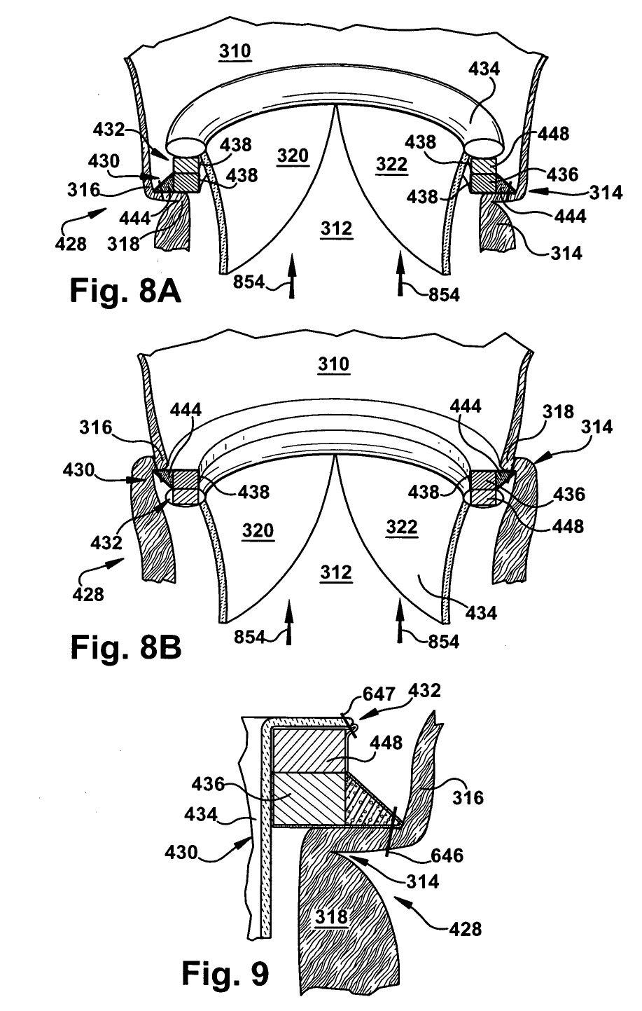

The first ring assembly 430 should be attached to the annulus 314 before being mated with the second ring assembly 432. Since the prosthetic cardiac valve 434 is carried by the second ring assembly 432, the magnetic mating between the first and second ring assemblies 430 and 432 acts to position and secure the prosthetic cardiac valve 434 to the annulus 314 of the native mitral valve 306. FIG. 8A depicts an assembled apparatus 428 according to a first configuration of the present invention, located within the annulus 314 of the native mitral valve 306. A cross-sectional view of this attachment, as installed in the annulus 314, is shown in FIG. 9.

FIG. 8B illustrates an apparatus 428 according to an alternate configuration of the present invention. In this arrangement, the second magnetic member 448 is located either within (as shown) or adjacent (not shown, but similar to the arrangement in FIG. 8A) the rim portion of the prosthetic cardiac valve 434. As shown in FIG. 8B, the first ring assembly 430 is attached to the inferior aspect 318 of the annulus 314, rather than to the superior aspect 316 as with the assembly of FIG. 8A. The attachment of the first ring assembly 430 to the annulus 314 may be done in much the same manner as with the previously described assembly, however, using sutures 646 or other suitable attachment means.

When the prosthetic cardiac valve 434 of the second embodiment is appropriately positioned adjacent the first ring assembly 430, the second magnetic member 448 may be magnetically attracted to the first magnetic member 436 of the first ring assembly to sealingly attach the first ring assembly to the prosthetic cardiac valve. However, the prosthetic cardiac valve 434 of FIG. 8B is suspended from the annulus 314 as opposed to the prosthetic cardiac valve 434 of FIG. 8A. The leaflets 320 and 322 extend through the annulus 314 in the arrangement of FIG. 8A, while the leaflets 320 and 322 are spaced apart from the annulus 314 by the first magnetic member 436 in the arrangement of FIG. 8B.

This relationship between the leaflets 320 and 322 and the annulus 314 emphasizes the difference between the configurations shown in FIGS. 8A and 8B. These configurations may be suited to replace different valves within a human heart and may, therefore, have different structural requirements. For example, the configuration shown in FIG. 8A is installed within the annulus 314 of a native mitral valve 306. The pressure arrows 854 show how the pressure gradient between the left ventricle 312 and left atrium 310 tends to push the prosthetic cardiac valve 434 away from the annulus 314. Therefore, when an apparatus 428 in the configuration of FIG. 8A is used to replace a native mitral valve 306, the first and second magnetic members 436 and 448 must be chosen to have an attraction strength sufficient to overcome the pressure gradient at the annulus 314 of the native mitral valve 306.

In contrast, FIG. 8B depicts an apparatus 428 located below the annulus 314 of the native mitral valve 306. The pressure arrows 854 show how the pressure gradient between the left ventricle 312 and the left atrium 310 tend to push the prosthetic cardiac valve 434 toward the annulus 314. Therefore, when an apparatus 428 as shown in FIG. 8B is used to replace a native mitral valve, the first and second magnetic members 436 and 448 may have a lower magnetic attraction than in the mitral valve example given with respect to FIG. 8A because the pressure gradient (shown by arrows 854) in this orientation helps to urge the first and second ring assemblies 430 and 432 together.

While the pressure gradient of the configuration depicted in FIG. 8B may seem to have advantages not provided by the configuration of FIG. 8A, the annulus 314 of FIG. 8B may not always be readily accessible for installation as shown. The choice of which apparatus 428 of the various configurations of the present invention to use in a particular application may therefore be made by one of ordinary skill in the art considering factors such as the relative strengths of the magnetic members 436 and 438, the location within the heart at which the apparatus is to be installed, and ease of access to either the superior or inferior aspects of the subject annulus. In addition, apparatus 428 having the configurations of FIGS. 8A and 8B are not restricted to use in the mitral position, but may be used to replace any cardiac valve as desired.

The first and second magnetic members 436 and 448 of the present invention may comprise a ring, wire, or band made of a material capable of producing a magnetic field. Alternatively, the first and second magnetic members 436 and 448 may comprise a plurality of magnets (not shown) arranged in a rigid or flexible housing, such as the sewing ring 444, or merely enclosed within the biocompatible material 438. Examples of suitable materials include NdFeB (neodymium iron boron), SmCo (samarium cobalt), and Alnico (aluminum nickel cobalt). The magnetic force exerted by the first and second magnetic members 436 and 448 will depend on various factors, including the materials used and the size of the first and second magnetic members. In addition, different applications of the present invention will call for different forces to be exerted between the first and second magnetic members 436 and 448. For instance, application of the first ring assembly 430 to a patient's mitral valve 306 may call for a lesser or greater force as compared to application of the first ring assembly to a patient's tricuspid valve (not shown).

The biocompatible material 438 covering the first and second magnetic members 436 and 448 of any embodiment of the present invention may be the same or different materials. The biocompatible material 438 may be any suitable arrangement of a rigid or flexible synthetic material such as stainless steel, titanium, Dacron®, woven velour, polyurethane, PTFE, ePTFE, heparin-coated fabric, or a combination thereof. Alternatively or additionally, the biocompatible material 438 may be at least partially comprised of a biological material such as animal pericardium; animal peritoneum; a homograft; a patient graft; a cell-seeded tissue; or any other animal, human, or laboratory-grown tissue. The biocompatible material 438 may also include additional features (not shown), such as loops or barbs, to facilitate attachment of the biocompatible material to the annulus 314 of the native mitral valve 306.

To replace a patient's native mitral valve 306 with the present invention, a physician must first access the mitral valve. One means of accessing the mitral valve 306 is to use a transthoracic approach and create an incision or port on the heart wall. Once the mitral valve 306 has been accessed, the physician may then determine the dimensions of the mitral valve. Various devices and methods for determining the dimensions of a cardiac valve are known in the art. The physician may also or instead determine the dimensions of the mitral valve 306 prior to surgery by using fluoroscopic and/or echocardiographic data.

After sizing the mitral valve 306, the physician may then select an appropriately-sized apparatus 428 of a chosen embodiment of the present invention for replacement of the mitral valve. More particularly, the physician may select a first ring assembly 430 having a size and shape complementary to the superior aspect 316 of the annulus 314 of the mitral valve 306. Similarly, the physician may select a prosthetic cardiac valve 434 carried by a second ring assembly 432 having a size and shape complementary to the first ring assembly 430. After selecting an appropriately-sized apparatus 428, the physician may excise the native mitral valve leaflets 320 and 322 or otherwise remove or deactivate the native mitral valve 306.

Next, the physician may secure the first ring assembly 430 to the superior aspect 316 of the annulus 314 of the mitral valve 306. The first ring assembly 430 can be attached to the annulus 314 of the mitral valve 306, for example, by threading sutures 646 through the sewing ring 444 of the first ring assembly 430 and then stitching the first ring assembly to the annulus of the mitral valve. Alternatively, the first ring assembly 430 may be attached to the annulus 314 of the mitral valve 306 by gluing, pinning, clamping, or any other suitable attachment method.

After securing the first ring assembly 430 to the superior aspect 316 of the annulus 314 of the mitral valve 306, the physician may then deliver the second ring assembly 432, which carries the prosthetic cardiac valve 434, to the annulus 314. The physician may position the second ring assembly 432 adjacent the first ring assembly 430 so that the second magnetic member 448 of the second ring assembly is magnetically attracted to the first magnetic member 436 of the first ring assembly. Consequently, the first and second ring assemblies 430 and 432 are pulled toward one another and sealingly attach to form a functional replacement mitral valve 306.

The need may arise to replace a previously implanted prosthetic cardiac valve 434, because prosthetic cardiac valves, and especially bioprosthetic cardiac valves, typically deteriorate over time. More particularly, where a patient's native mitral valve 306 has been previously replaced with an apparatus 428 according to the present invention, and a previously implanted prosthetic cardiac valve 434 secured in a second ring assembly has deteriorated, the physician may use a replacement prosthetic cardiac valve secured in a second ring assembly 432 to restore the normal function of the replacement mitral valve without disturbing the implanted first ring assembly 430 from the initial replacement procedure.

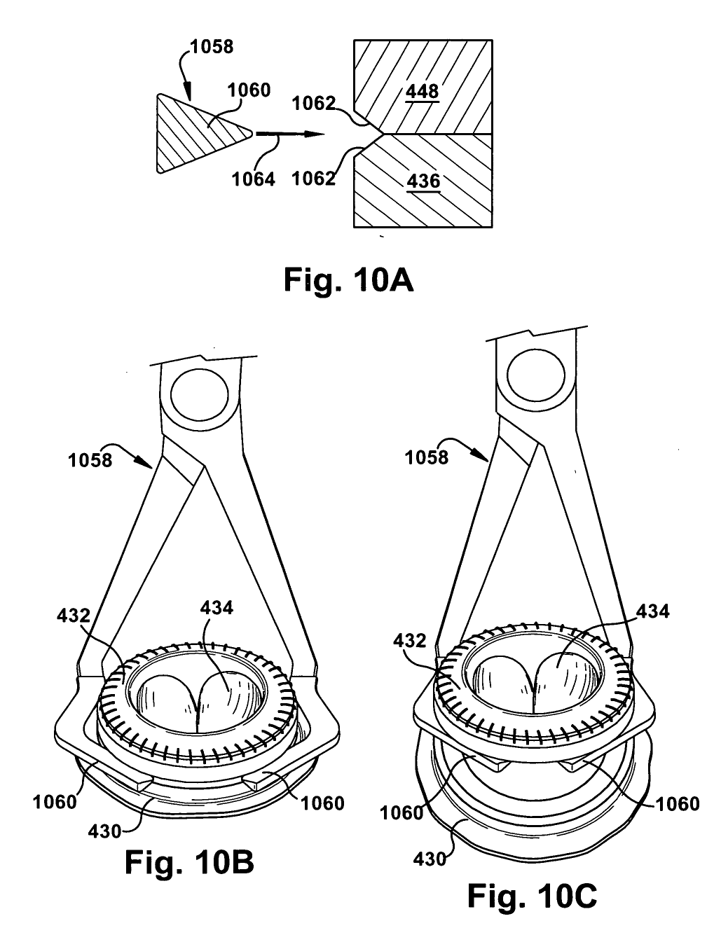

The physician may use a transthoracic approach to replace the previously implanted prosthetic cardiac valve 434 secured in a second ring assembly 432. For example, the physician may first gain access to the previously implanted prosthetic cardiac valve 434 secured in a second ring assembly 432 by creating an incision or port on the heart wall. After accessing the site of the previously implanted prosthetic cardiac valve 432, the physician may detach the previously implanted second ring assembly 432 from a previously implanted first ring assembly 430 by separating the previously implanted second ring assembly 432 such that the second magnetic member 448 of the previously implanted second ring assembly 432 is no longer magnetically attracted to the first magnetic member 436 of the previously implanted first ring assembly 430. This may be done, for instance, with the separation tool 1058 depicted in FIGS. 1A, 10B, and 10C.

FIG. 10A shows a magnified schematic view of a blade 1060 of the separation tool 1058 in relation to the first and second magnetic members 436 and 448 (the prosthetic cardiac valve 434, biocompatible material 438, and sewing ring 444, if present, have been omitted from this Figure for clarity). When the depicted separation tool 1058 is used, the first and second magnetic members 436 and 448 should each be provided with a beveled edge 1062 on the mating surfaces thereof. The blade 1060 can then be inserted into the beveled edges 1062 and move laterally toward the interface between the first and second magnetic members 436 and 448, in the direction of the separation arrow 1064. The wedging action of the blade 1060 against the beveled edges 1062 of the first and second magnetic members 436 and 448 acts to initially separate the first and second magnetic members. The first and second magnetic members 436 and 448 separate as the thickness of the blade 1060 is inserted therebetween, and the previously implanted second ring assembly 432, with prosthetic cardiac valve 434 attached, may be removed from the apparatus 428.

The operation of the separation tool 1058 is shown pictorially in the sequence of FIGS. 10B-10C. The separation tool 1058 of the Figures is a forceps-type tool having blades 1060 adapted to mate with the contour of the first and second ring assemblies 430 and 432, with the blades moved by a scissors-like action of the separation tool. Many different configurations of a separation tool 1058 are possible, and a suitable separation tool 1058 can be readily designed for a desired application by one of ordinary skill in the art.

Regardless of the mechanism of removal of the previously implanted second ring assembly 432 and attached prosthetic cardiac valve 434, the physician next positions the replacement prosthetic cardiac valve 434 secured in a second ring assembly 432 adjacent the previously implanted first ring assembly 430. Consequently, the second magnetic member 448 of the replacement second ring assembly 432 is magnetically attracted to the first magnetic member 436 of the previously implanted first ring assembly 430 so that the previously implanted first ring assembly 430 is sealingly attached to the replacement prosthetic cardiac valve 434 and a functional replacement mitral valve 306 is produced.

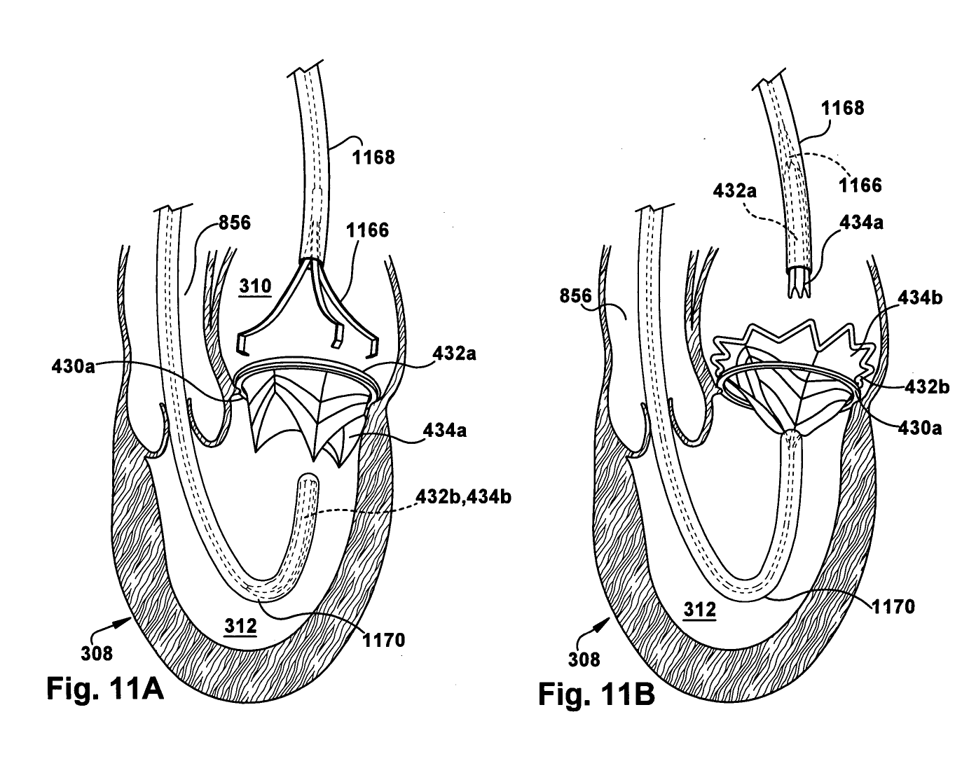

Alternatively to the previously described procedure, a physician may remove a previously implanted prosthetic cardiac valve 434a secured in a second ring assembly 432a using a percutaneous approach, as shown in FIGS. 11A and 11B. For example, the previously implanted second ring assembly 432a may first be removed while the previously implanted first ring assembly 430a remains attached to the annulus 314 of the mitral valve 306. As shown in FIG. 11A, this could be accomplished using a collapsible second ring assembly 432a and a plurality of hooks 1166, the hooks being advanced to a position adjacent the second ring assembly 432a through a removal catheter 1168. The removal catheter 1168 may be advanced through any suitable vascular structure until reaching a position adjacent the desired replacement valve site. For instance, and as shown in FIGS. 11A and 11B, the physician may insert the removal catheter 1168 into either the right or left jugular vein (not shown), a femoral vein (not shown), or the subclavian vein (not shown) using a known percutaneous technique, such as the Seldinger technique. The hooks 1166 are manipulated to snag the second ring assembly 432a and exert sufficient pull to separate the second ring assembly 432a from the first ring assembly 430a by overcoming the magnetic attraction therebetween. The hooks 1166 are then withdrawn into the removal catheter 1168, into the position shown in FIG. 11B, pulling the flexible second ring assembly 432a and attached prosthetic cardiac valve 434a into the removal catheter 1168 for removal from the patient's heart 308.

The replacement prosthetic cardiac valve 434b secured in a second ring assembly 432b may then be introduced via an introduction catheter 1170. The introduction catheter 1170 may be advanced through any suitable vascular structure until reaching a position adjacent the desired replacement valve site. For instance, and as shown in FIGS. 11A and 11B, the physician may insert the introduction catheter 1170 into a femoral artery (not shown) and then advance the catheter in a retrograde fashion through the aorta 856, into the left ventricle 312, and up through the mitral valve 306 into the left atrium 310. The replacement prosthetic cardiac valve 434b secured in a second ring assembly 432b may be deployed from the introduction catheter 1170 and positioned adjacent the previously implanted first ring assembly 430a. The deployment of the second ring assembly 432b and associated prosthetic cardiac valve 434b from the introduction catheter 1170 may be accomplished in any suitable manner. For example, the second ring assembly 432b could be a flexible, self-expanding ring 432b and could splay outward into an expanded condition upon exiting the introduction catheter 1170. When the second ring assembly 432b is flexible, the first ring assembly 430a could help provide structural support to the second ring assembly 432b once the first and second ring assemblies are magnetically coupled together.

After the replacement prosthetic cardiac valve 434b secured in a second ring assembly 432b is positioned adjacent the previously implanted first ring assembly 430a, the second magnetic member 448b of the replacement second ring assembly 432b is magnetically attracted to the first magnetic member 436a of the previously implanted first ring assembly 430a. Consequently, the replacement prosthetic cardiac valve 434b becomes sealingly attached to the previously implanted first ring assembly 430a and a functional replacement mitral valve 306 is reformed. Temporary circulatory assist may be beneficial during this procedure.

While aspects of the present invention have been particularly shown and described with reference to the preferred embodiment above, it will be understood by those of ordinary skill in the art that various additional embodiments may be contemplated without departing from the spirit and scope of the present invention. For example, one of the first and second magnetic members 436 and 448 could be made of a magnetic material with the other one of the first and second magnetic members being an inert material (such as a metal) attracted by the magnetic material. Any of the structures of the apparatus 428 could be made of any suitable material or combination of materials, and in any desired configuration or orientation. The biocompatible material 438 could play some structural role in supporting, implanting, and/or removing the first or second ring assemblies 430 or 432. The second ring assembly 432 could be formed integrally with the prosthetic cardiac valve 434 or could provide some structural support for at least one valve leaflet or other portion of the prosthetic cardiac valve. The separation tool 1058 could have the structure of a noose, tongs, scissors, chisel, hook, finger, or any other suitable configuration. Separation can also be achieved by de-magnetizing the first and second magnetic members 436 and 448. The apparatus 428 could be initially implanted and/or reaccessed either percutaneously (using minimally invasive surgery techniques) or through a transthoracic or other “open” approach. The prosthetic cardiac valve 434, first ring assembly 430, and/or second ring assembly 432 could be fastened to the papillary muscles 326 either directly or through the native or prosthetic chordae tendineae 324. The first ring assembly 430 could be fastened to the annulus 314 with sutures, adhesives, staples, barbs, anchors, biological ingrowth, or any other suitable attachment means. A replacement prosthetic cardiac valve 434b could be of a different type than the previously implanted prosthetic cardiac valve 434a. A device or method incorporating any of these features should be understood to fall under the scope of the present invention as determined based upon the claims below and any equivalents thereof.

Other aspects, objects, and advantages of the present invention can be obtained from a study of the drawings, the disclosure, and the appended claims.

Having described the invention, we claim:

1. An apparatus for replacing a cardiac valve, said apparatus comprising:
a first ring assembly including a first magnetic member;
a second ring assembly including a second magnetic member and being magnetically attachable to the first magnetic member to sealingly attach the first and second ring assemblies together; and
a prosthetic cardiac valve secured within the second ring assembly.

2. The apparatus of claim 1, wherein at least one of the first and second magnetic members is at least partially covered with a biocompatible material.

3. The apparatus of claim 1, wherein the prosthetic cardiac valve has a valve annulus, the first ring assembly being attachable to the valve annulus.

4. The apparatus of claim 3, wherein the first ring assembly includes a sewing ring adapted for attachment to the valve annulus.

5. The apparatus of claim 1, wherein the prosthetic cardiac valve has at least two valve leaflets, the first ring assembly defines a first ring assembly annulus, and at least a portion of a valve leaflet extends through the first ring assembly annulus.

6. The apparatus of claim 1, wherein the prosthetic cardiac valve is formed integrally with the first ring assembly.

7. The apparatus of claim 1, wherein the first and second ring assemblies each include a beveled edge, and the beveled edges are adjacent each other when the first and second ring assemblies are sealingly attached together.

8. An apparatus for replacing a cardiac valve having a valve annulus, the valve annulus having a superior aspect and an inferior aspect, the apparatus comprising:
a first ring assembly including a first magnetic member and being attachable to the valve annulus;
a second ring assembly including a second magnetic member and being magnetically attachable to the first magnetic member to sealingly attach the first and second ring assemblies together and a prosthetic cardiac valve secured within the second ring assembly.

9. The apparatus of claim 8, wherein the first ring assembly is attachable to the superior aspect of the valve annulus.

10. The apparatus of claim 8, wherein the first ring assembly is attachable to the inferior aspect of the valve annulus.

11. The apparatus of claim 8, wherein at least one of the first and second magnetic members is at least partially covered with a biocompatible material.

12. The apparatus of claim 8, wherein the first ring assembly includes a sewing ring adapted for attachment to the valve annulus.

13. The apparatus of claim 8, wherein the prosthetic cardiac valve has at least two valve leaflets, the first ring assembly defines a first ring assembly annulus, and at least a portion of a valve leaflet extends through the first ring assembly annulus.

14. The apparatus of claim 8, wherein the prosthetic cardiac valve is formed integrally with the first ring assembly.

15. The apparatus of claim 8, wherein the first and second ring assemblies each include a beveled edge, and the beveled edges are adjacent each other when the first and second ring assemblies are sealingly attached together.

16. A method for replacing a native cardiac valve within a valve annulus, the method comprising the steps of:
removing the native cardiac valve from the valve annulus;
providing a first ring assembly attachable to the valve annulus, the first ring assembly including a first magnetic member;
providing a second ring assembly including a second magnetic member, the second ring assembly including a prosthetic cardiac valve secured thereto;
securing the first ring assembly to the valve annulus; and
moving the second ring assembly into magnetic engagement with the first ring assembly to position the prosthetic cardiac valve into a predetermined relationship within the valve annulus.

17. The method of claim 16, wherein at least one of the first and second magnetic members is at least partially covered with a biocompatible material.

18. The method of claim 16, wherein the step of securing the first ring assembly to the valve annulus includes the step of suturing the first ring assembly to the valve annulus.

19. The method of claim 16, wherein the prosthetic cardiac valve has at least two valve leaflets, and the step of moving the second ring assembly into magnetic engagement with the first ring assembly to position the prosthetic cardiac valve into a predetermined relationship with the valve annulus includes the step of extending at least a portion of a valve leaflet through the valve annulus.

20. The method of claim 16, wherein the second ring assembly is an initial second ring assembly, and further including the steps of:
providing a replacement second ring assembly including a replacement second magnetic member, the replacement second ring assembly including a replacement prosthetic cardiac valve secured thereto;
separating the initial second ring assembly from the first ring assembly; and
moving the replacement second ring assembly into magnetic engagement with the first ring assembly to position the replacement prosthetic cardiac valve into a predetermined relationship with the valve annulus.

21. The method of claim 20, wherein the first and initial second ring assemblies each include a beveled edge, and the beveled edges are adjacent each other when the first and initial second ring assemblies are in magnetic engagement, and the step of separating the initial second ring assembly from the first ring assembly includes the steps of:
providing a separation tool;
engaging the beveled edges with the separation tool; and
breaking the magnetic engagement between the first ring assembly and the initial second ring assembly.
